(19) United States
(12) Patent Application Publication
(10) Pub. No.: US 2021/0299098 A1
(43) Pub. Date: Sep. 30, 2021

(54) SELECTIVE ESTROGEN RECEPTOR MODULATOR FOR TREATMENT OF PANCREATIC CANCER

(71) Applicant: CIPLA LIMITED, Mumbai (IN)

(72) Inventors: Kalpana JOSHI, Mumbai (IN); Jeevan GHOSALKAR, Mumbai (IN); Vinay SONAWANE, Mumbai (IN)

(21) Appl. No.: 17/216,756

(22) Filed: Mar. 30, 2021

(30) Foreign Application Priority Data
Mar. 31, 2020 (IN) 202021014204

(51) Int. Cl. A61K 31/4025; A61K 45/06; A61K 31/138; A61P 35/04

(52) U.S. Cl. CPC A61K 31/4025; A61P 35/04; A61K 31/138; A61K 45/06

(57) ABSTRACT

Disclosed is a method for treating of cancer, specifically pancreatic cancer by administering a selective estrogen receptor modulator (SERM), e.g., levormeloxifene, alone or in combination with one or more additional cancer therapies.

### CROSS-REFERENCE TO RELATED APPLICATIONS

This application claims the benefit of Indian Provisional Patent Application Serial Number 202021014204 filed on Mar. 31, 2020, the contents of which are hereby incorporated in its entirety.

### FIELD OF INVENTION

The present invention relates to a method of treatment of cancer, specifically pancreatic cancer. In particular, the present invention pertains to methods for the treatment of pancreatic cancer by administering a selective estrogen receptor modulator (SERM) alone or in combination with one or more additional cancer therapies.

### BACKGROUND OF INVENTION

Pancreatic cancer is a malignant neoplasm originating from transformed cells arising in tissues forming the pancreas. Pancreatic cancer is one of the most common causes of cancer-related deaths in the world. Due to the absence of specific symptoms, the lack of early detection techniques, and highly aggressive phenotypes, pancreatic cancer is usually diagnosed at an advanced-incurable and metastatic stages. Thus, pancreatic cancer has an extremely poor prognosis: for all stages combined, the 1- and 5-year relative survival rates are 25% and 6%, respectively.

Approximately 20% of patients are diagnosed with localized and potentially curable tumors. However, majority (95%) of the cases of pancreatic cancer are adenocarcinomas which resemble the pancreatic ductal cell. Metastasis of this cancer is generally local, most often involving the liver, lung, spleen, lymphatic system, adrenal glands and transverse colon.

Pancreatic cancer is treated in several ways, either alone or in combination based upon the stages of malignancy. Currently, surgery or resection is still the most basic means for treating pancreatic cancer for curative treatment. However, despite the fact that surgical options for pancreatic cancer are now associated with acceptable outcomes, they often prove ineffective in controlling the disease with reported recurrence rates approaching almost 80% (both locally and distant) and a 5-year survival rate of only 10%-24% for cases involving total resection.

Other treatment options of pancreatic cancer range from systemic chemotherapy alone to combined forms of treatment with chemoradiation and chemotherapy. Chemotherapy treatments can be categorized as adjuvant (treatment after surgery), neo-adjuvant (treatment prior to surgery) and palliative. The most common chemotherapeutic agents that are used to treat pancreatic cancer are gemcitabine, 5-fluouracil, capecitabine, cisplatin and oxaliplatin. These agents function on the basis of cross-linking mechanisms in which their reactive region interacts with the cell's DNA or RNA nucleotides, thus disrupting the cell cycle progression which leads to cancer cell apoptosis.

Further, targeted therapy of erlotinib in combination with gemcitabine has been approved for patients with advanced pancreatic cancer. Targeted therapy is a treatment that targets the cancer's specific genes, proteins or the tissue environment that contribute to cancer growth and survival. This type of treatment blocks the growth and spread of cancer cells while limiting damage to healthy cells.

Although some progress has been made in the management of pancreatic cancer over the years, the benefits from such treatments are considerably small and have always been confined to a minority of the treated population. Further, aggressive tumor biology and limited efficacy of conventional therapies have led to rapid progression of this disease as well as an increase in the cancer specific mortality rate. Hence, there still remains a need to develop different treatment approaches in a cost effective and a time efficient manner.

Estrogen-binding sites are present in tissue samples of adenocarcinoma of the pancreas. The sex steroid estrogen, androgen and progestin hormones all play a functional role in the healthy pancreas. Pancreatic cancer, a highly lethal disease, has a higher prevalence in men compared to women. Selective estrogen receptor modulators (SERMs) are synthetic molecules which bind to estrogen receptors and can modulate its transcriptional capabilities in different ways in diverse estrogen target tissues. While these factors may suggest a potential role for sex hormone regulation or dysregulation in pancreatic carcinogenesis, the majority of epidemiological studies have failed to support an association between hormonal factors and pancreatic cancer development. Similarly, hormonal manipulation with the antiestrogen tamoxifen as a therapeutic strategy for pancreatic cancer has been explored clinically, but was met with limited success. Unfortunately, use of tamoxifen is associated with de-novo and acquired resistance and some undesirable side effects.

Centchroman (INN: ormeloxifene), is a non-steroidal Selective Estrogen Receptor Modulator (SERM) used as oral contraceptive (half-life of about 168 hours). Besides contraception, this SERM is also clinically useful in the management of mastalgia, fibroadenoma and has promising therapeutic efficacy in a variety of cancers. Due to estrogenic activity, this drug also has anti-osteoporotic and cardioprotective activity. The commercial ormeloxifene hydrochloride is a 1:1 (w/w) mixture of its two optical isomers (D- and L-). According to literature L-form has about 7 times higher receptor affinity than the D-isomer. The ability of ormeloxifene to inhibit rapid cell proliferation in the endometrium during embryonic implantation along with its favorable bioavailability, stability and safety in humans makes it an attractive repurposing molecule for controlling undesired rapid cell proliferation such as endometriosis and cancerous conditions. Accordingly, the anti-cancer activity of ormeloxifene has been checked in several cancers including prostate cancer, ovarian cancer, CML, breast cancer etc. But no attempt has been made to study the anti-cancer activity of levormeloxifene.

The inventors of the present invention have found that levormeloxifene exhibits a significant role in the inhibition of pancreatic tumor growth, progression and metastasis.

### SUMMARY OF THE INVENTION

Disclosed herein are methods of treating pancreatic cancer by administering the active agent acting as selective estrogen receptor modulator (SERM). The active agent may be administered in combination with one or more additional cancer therapies. In some instances, the active agent may be administered in combination with one or more cancer chemotherapeutic agents.

Also disclosed herein are pharmaceutically acceptable compositions containing selective estrogen receptor modulator, in some cases in combination with one or more anti-cancer drugs for the treatment of pancreatic cancer.

The details of one or more embodiments are set forth in the descriptions below. Other features, objects, and advantages will be apparent from the description and from the claims.

### BRIEF DESCRIPTION OF THE FIGURES

FIG. 1 depicts a concentration-effect curve exhibiting efficacy of levormeloxifene (“L-Cent”) and tamoxifen (“TAM”) in pancreatic cancer cell lines AsPc1.

FIG. 2 depicts a concentration-effect curve exhibiting efficacy of levormeloxifene (“L-Cent”) and tamoxifen (“TAM”) in pancreatic cancer cell lines BxPC3.

FIG. 3 depicts a concentration-effect curve exhibiting efficacy of levormeloxifene (“L-Cent”) and tamoxifen (“TAM”) in pancreatic cancer cell lines Miapaca2.

FIG. 4 depicts a concentration-effect curve exhibiting efficacy of Levormeloxifene (“L-Cent”) and tamoxifen (“TAM”) in pancreatic cancer cell lines Panc10.05.

FIG. 5 depicts a concentration-effect curve exhibiting efficacy of levormeloxifene (“L-Cent”) and tamoxifen (“TAM”) in pancreatic cancer cell lines SW1990.

FIG. 6 depicts a concentration-effect curve exhibiting efficacy of levormeloxifene (“L-Cent”) and tamoxifen (“TAM”) in pancreatic cancer cell lines Panc1.

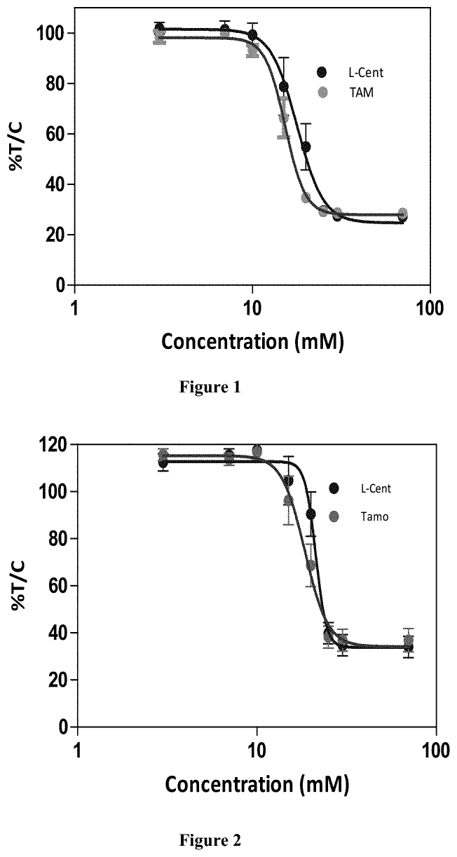

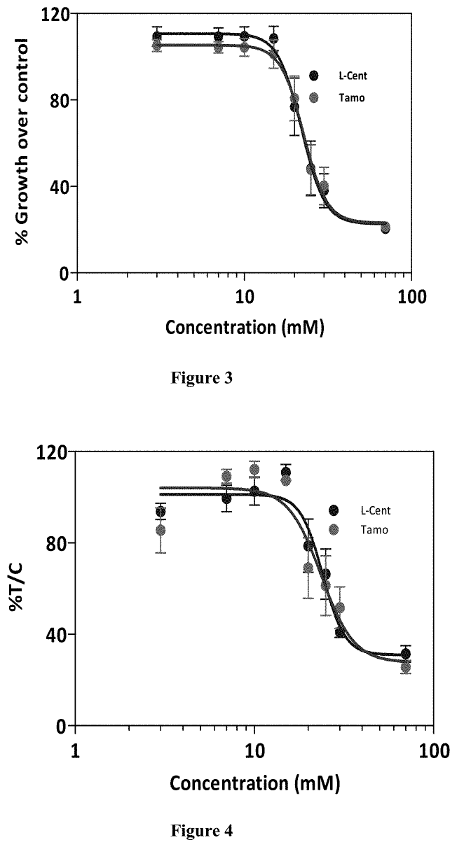

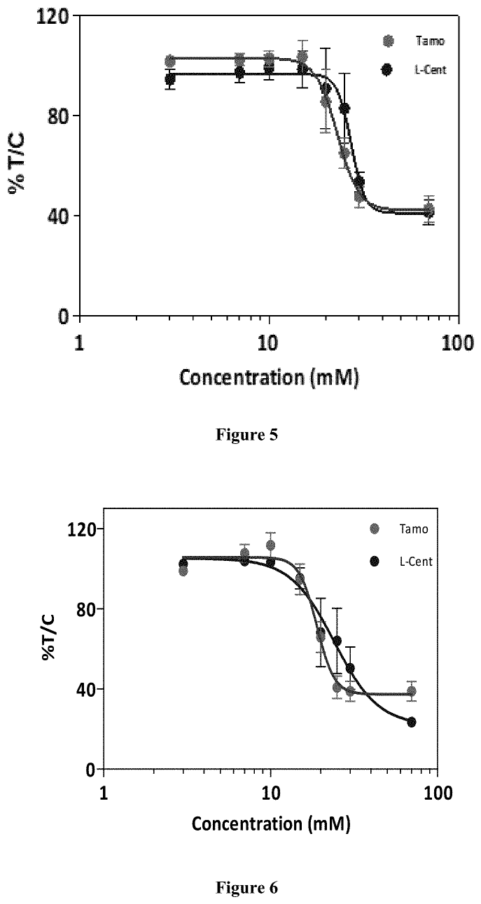

### DETAILED DESCRIPTION OF THE INVENTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. “Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Selective estrogen receptor modulators” or “SERMs” as used interchangeably herein refers to a compound that interacts with an ER and whose relative agonist/antagonist activities are manifest in a cell selective manner. The prevention of estrogen binding to the estrogen receptor may lead to decreased proliferation of estrogen dependent cancer cells. Levormeloxifene (NNC 46-0020, (−)-3,4-trans-7-methoxy-2,2-dimethyl-3-phenyl-4-[4-[2-(pyrrolidin-1-yl)ethoxy] phenyl]chromane) is a selective estrogen receptor modulator (SERM). It is the (−)-enantiomer of ormeloxifene which, under the name Centchroman, has been on the market in India as an oral contraceptive since the late 1980s and is presently under development in India for the treatment of advanced breast cancer.

Example 1 of U.S. Pat. No. 4,447,622 describes the preparation of the levormeloxifene. The structure of levormeloxifene is:

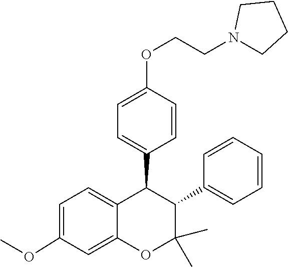 [SMILES: COc1ccc2c(c1)OC(C)(C)[C@@H](c1ccccc1)[C@@H]2c1ccc(OCCN2CCCC2)cc1]

In example 2 of U.S. Pat. No. 4,447,622, levormeloxifene is obtained as the free base and the hydrochloride salt.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like. Pharmaceutically acceptable and non-pharmaceutically acceptable salts may be prepared using procedures well known in the art, for example, by reacting a sufficiently basic compound such as an amine with a suitable acid comprising a physiologically acceptable anion.

In a preferred embodiment the active ingredient is levormeloxifene or a pharmaceutically acceptable salt thereof, more preferred levormeloxifene hydrogen chloride, levormeloxifene hydrogen fumarate, or levormeloxifene hydrogen maleate, most preferred levormeloxifene hydrogen fumarate.

There is provided a method of alleviating or treating pancreatic cancer by administration of levormeloxifene or its salt thereof optionally in combination with one or more anti-cancer drugs either simultaneously, sequentially, or separately. The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. As used herein, the term “delay” refers to methods that reduce the probability of disease development/extent in a given time frame, when compared to otherwise similar methods that do not include the use of Levormeloxifene or its pharmaceutically acceptable salts thereof. Probabilities can be established using clinical trials, but can also be determined using in vitro assays when correlations have been established.

In certain embodiments, administration of levormeloxifene or its pharmaceutically acceptable salts thereof can reduce tumor volume. For instance, levormeloxifene or its pharmaceutically acceptable salts thereof can be administered in an amount effective to reduce pancreatic tumor volume by at least 10%, 20%, 30%, 40%, 50%, 60%, 70%, 80%, or 90%. In some embodiments, levormeloxifene or its pharmaceutically acceptable salts thereof can inhibit pancreatic cancer cell proliferation. For instance, levormeloxifene or its pharmaceutically acceptable salts thereof can be administered in an amount effective to inhibit at least about 10%, 20%, 30%, 40%, 60%, 70%, 80%, 90%, or 100% of cell proliferation. In some embodiments, levormeloxifene or its salt thereof can inhibit pancreatic cancer metastasis. For instance, levormeloxifene or its pharmaceutically acceptable salts thereof can be administered in an amount effective to inhibit at least about 10%, 20%, 30%, 40%, 60%, 70%, 80%, 90%, or 100% of metastasis.

Levormeloxifene or its pharmaceutically acceptable salts thereof can be administered according to various dosing regimens. For instance, levormeloxifene or its pharmaceutically acceptable salts thereof can be administered once a day, twice a day, three times per day, or even more than three times a day. The levormeloxifene or its pharmaceutically acceptable salts thereof can be administered such that the total daily dose is at least 50 mg, at least 100 mg, at least 250 mg, at least 500 mg, at least 750 mg, at least 1,000 mg, at least 1,250 mg, at least 1,500 mg, at least 1,750 mg, or at least 2,000 mg. In some instances, the total daily dose can be from 5-5,000 mg, 10-5,000 mg, 25-5,000 mg, 50-5,000 mg, 100-5,000 mg, 200-2,500 mg, 500-2,500 mg, 10-2,500 mg, 50-2,500 mg, 100-2,500 mg, 100-2,000 mg, 250-2,000 mg, or 500-2,000 mg. In other embodiments, levormeloxifene or its pharmaceutically acceptable salts thereof can be administered less than once daily, instance, once every two days, once every three days, once every five days, once every seven days, once every ten days, once every fourteen days, once every twenty-eight days or once every month.

Levormeloxifene or its pharmaceutically acceptable salts thereof can be administered by other intermittent therapies as well, for instance 2 days on, 1 day off, e.g. “2/1.” Other on/off sequences that can be employed include 3/1; 3/2; 4/1; 5/1; 5/2; 5/3; 5/4; 6/1; 6/5; 7/1; 7/2; 7/3; 7/4; 7/5; 7/6; 8/1; 8/3; 8/5; 8/7; 9/1; 9/2; 9/4; 9/5; 9/7; 9/8; 10/1; 10/3; 10/7; and 10/9.

According to the present invention, there is provided a use of levormeloxifene or its pharmaceutically acceptable salts thereof in combination with one or more anti-cancer drugs either simultaneously, sequentially, or separately for the treatment of pancreatic cancer. In certain cases, levormeloxifene or its pharmaceutically acceptable salts thereof can be administered for a period of at least 1 week, at least 2 weeks, at least 4 weeks, at least 6 weeks, at least 8 weeks, or at least 10 weeks, prior to commencing treatment with additional agents. In some instances levormeloxifene or its pharmaceutically acceptable salts thereof and the other agent can be administered intermittently, for instance a period of levormeloxifene or its pharmaceutically acceptable salts thereof administration, followed by a period in which the other agent to administered, followed by another period of levormeloxifene or its pharmaceutically acceptable salts thereof administration. The cycle can be repeated as many times as necessary.

In certain cases, the combination of levormeloxifene or its pharmaceutically acceptable salts thereof and additional agent will exhibit a greater than additive effect (i.e., a synergistic effect). In other instance, the use of levormeloxifene or its pharmaceutically acceptable salts thereof permits a reduced amount of the other agent to be administered, without a corresponding decrease in therapeutic efficiency.

Preferably, one or more anti-cancer drugs that may be envisaged under the scope of the present invention may comprise currently approved targeted anti-cancer drugs for the treatment of pancreatic cancer such as, but not limited to, a nucleoside analogue, antifolate, antimetabolite, topoisomerase I inhibitor, anthracycline, podophyllotoxin, taxanes, vinca alkaloid, alkylating agent, platinum compound, proteasome inhibitor, nitrogen mustard, estrogen analogue, monoclonal antibody, tyrosine kinase inhibitor, mTOR inhibitor, retinoid, immunomodulatory agent, histone deacetylase inhibitor, other kinase inhibitor, metabolic inhibitors, and microtubule inhibitors. Exemplary anti-cancer drugs that can be used in combination with levormeloxifene or its pharmaceutically acceptable salts thereof include tamoxifen, everolimus, erlotinib, gemcitabine, 5-fluorouracil, capecitabine, cisplatin, paclitaxel and oxaliplatin. In certain cases, the anti-cancer drug is gemcitabine. In other embodiments, the anti-cancer drug is tamoxifen.

In certain embodiments, levormeloxifene is administered as a single agent, i.e., without concomitant administration of any other therapy. In other embodiments, levormeloxifene is administered in combination with one or more of the above enumerated drugs or drug classes, but not in combination with any other therapeutic agent.

In cases of combination therapy, it is possible that a unitary dosage form comprising both levormeloxifene or its pharmaceutically acceptable salts thereof and one or more additional anti-cancer drugs may be employed. In some instances, the combinations may be provided in form of kit preparation wherein levormeloxifene or its pharmaceutically acceptable salts thereof is present in an oral or parenteral composition and the additional anti-cancer drug therapy may be provided in an oral or parenteral composition. In one embodiment, the kit preparation may be provided in an all oral dosage form presentation wherein both the levormeloxifene or its pharmaceutically acceptable salts thereof and the additional anti-cancer drug are presented in an oral dosage form. In another embodiment, the kit preparation may be provided as an oral plus parenteral dosage form presentation wherein levormeloxifene or its pharmaceutically acceptable salts thereof is presented in an oral form and the additional anti-cancer drug is presented in a parenteral form. Alternatively, the kit preparation may be provided wherein levormeloxifene or its pharmaceutically acceptable salts thereof is presented in a parenteral form and the additional anti-cancer drug is presented in an oral dosage form.

In some instances, levormeloxifene or its pharmaceutically acceptable salts thereof can be used in combination with ionizing radiation, ablation therapy, embolization therapy, and/or surgical interventions for the treatment of pancreatic cancer. levormeloxifene or its pharmaceutically acceptable salts thereof can be administered before, during, or after treatment with ionizing radiation or surgical intervention. In certain cases, levormeloxifene or its pharmaceutically acceptable salts thereof can be administered for a period of at least 1 week, at least 2 weeks, at least 4 weeks, at least 6 weeks, at least 8 weeks, or at least 10 weeks, prior to commencing treatment with ionizing radiation or surgery. Exemplary forms of radiation include x-rays, gamma rays, electron beams and proton beams. Administration of levormeloxifene or its pharmaceutically acceptable salts thereof can permit a reduction in the total exposure of the patient to ionizing radiation, without a corresponding reduction in therapeutic efficiency. In certain instances, levormeloxifene or its pharmaceutically acceptable salts thereof can be administered both prior and subsequent to ionizing radiation and/or surgical interventions.

The present inventors have discovered the levormeloxifene or its pharmaceutically acceptable salts thereof is surprisingly effective for the treatment of pancreatic cancer at various stages. The TNM (tumor size/lymph node/metastasis) system is used in conjunction with a number to include the severity of the cancer; higher numbers indicate the cancer is more advanced. The TNM system includes the following stages of pancreatic cancer:

Stage 0: The tumor is confined to the top layers of pancreatic duct cells and has not invaded deeper tissues. It has not spread outside of the pancreas. These tumors are sometimes referred to as pancreatic carcinoma in situ or pancreatic intraepithelial neoplasia III (PanIn III).
Stage IA: The tumor is confined to the pancreas and is 2 cm across or smaller (T1). The cancer has not spread to nearby lymph nodes (N0) or distant sites (M0).
Stage IB: The tumor is confined to the pancreas and is larger than 2 cm across (T2). The cancer has not spread to nearby lymph nodes (N0) or distant sites (M0).
Stage IIA: The tumor is growing outside the pancreas but not into major blood vessels or nerves (T3). The cancer has not spread to nearby lymph nodes (N0) or distant sites (M0).
Stage IIB: The tumor is either confined to the pancreas or growing outside the pancreas but not into major blood vessels or nerves. The cancer has spread to nearby lymph nodes (N1) but not to distant sites (M0).
Stage III: The tumor is growing outside the pancreas and into nearby major blood vessels or nerves (T4). The cancer may or may not have spread to nearby lymph nodes (Any N). It has not spread to distant sites (M0).
Stage IV: The cancer has spread to distant sites (M1).

Levormeloxifene or its pharmaceutically acceptable salts thereof can be used to treat Stage 0 pancreatic cancer, Stage I pancreatic cancer, Stage II pancreatic cancer Stage III pancreatic cancer, or Stage IV pancreatic cancer. In certain embodiments, levormeloxifene can be used to treat at least Stage I pancreatic cancer (meaning any pancreatic cancer that has advanced to Stage I or greater severity of disease), at least Stage II pancreatic cancer, at least Stage III pancreatic cancer, or at least Stage IV pancreatic cancer. In certain embodiments, levormeloxifene can be used to treat patients having no greater than Stage III pancreatic cancer (that is, cancers excluding Stage IV), no greater than Stage II pancreatic cancer, or no greater than Stage I pancreatic cancer. In some embodiments, levormeloxifene or its pharmaceutically acceptable salts thereof can reduce tumor size, inhibit tumor growth, alleviate symptoms, delay progression, prolong survival, including, but not limited to disease free survival, prevent or delay pancreatic cancer metastasis, reduce or eliminate preexisting pancreatic cancer metastasis, and/or prevent recurrence of pancreatic cancer. In certain embodiments, levormeloxifene or its pharmaceutically acceptable salts thereof can be administered to a treatment-naive patient, i.e., a patient that has not yet undergone a course of therapy with a different chemotherapeutic agent.

In other embodiments, levormeloxifene or its pharmaceutically acceptable salts thereof can be administered to a patient who has already received one or more courses of chemotherapy. In some embodiments, the patient can have recurrent pancreatic cancer, or pancreatic cancer that is refractory to one or more therapies. In some embodiments, the one or more chemotherapies includes gemcitabine, for instance gemcitabine monotherapy. In some embodiments, the prior chemotherapy includes administration of gemcitabine and erlotinib, gemcitabine and capecitabine, gemcitabine and 5-FU, or gemcitabine, erlotinib, capecitabine, and/or 5-FU. In some embodiments, the prior therapy is adjuvant gemcitabine therapy. The prior chemotherapy can have been given for at least 1, 2, 3, 4, 5, 6, 7, 8, 9, 10, 11, or 12 months prior to commencement of levormeloxifene or its pharmaceutically acceptable salts thereof therapy. In certain embodiments, Levormeloxifene or its pharmaceutically acceptable salts thereof can be added to any of the foregoing treatment regimens without discontinuing the earlier treatment.

Preferably, levormeloxifene or its pharmaceutically acceptable salts thereof may be provided in the form of a pharmaceutical composition such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention. Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The inventors of the present invention have also found that the solubility properties of levormeloxifene or its pharmaceutically acceptable salts thereof may be improved by nanosizing thus leading to better bioavailability and dose reduction of the drug. For instance, levormeloxifene or its pharmaceutically acceptable salts thereof may be present in the form of nanoparticles which have an average particle size of less than 2,000 nm, less than 1,500 nm, less than 1,000 nm, less than 750 nm, less than 500 nm, or less than 250 nm.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: In Vitro 2-D Assay

The in vitro anti-tumor activity of levormeloxifene or its pharmaceutically acceptable salt was evaluated in six human pancreatic cancer cell lines: AsPc 1 (A), BxPC3 (B), Miapaca2 (C), Panc 10.05 (D), SW1990 (E) and Panc 1 (F).

AsPc 1 (A), BxPC3 (B), Miapaca2 (C), Panc 10.05 (D), SW1990 (E) and Panc 1 (F) were established at Oncotest from the corresponding human patient derived xenograft. CAPAN-2, HUP-T3 and PA-TU-8902 were purchased from DSMZ (Braunschweig, Germany). PANC-1 was purchased from CLS Cell Line Services GmbH (Eppelheim, Germany). BxPC-3, HPAC and MIA-Pa-Ca-2 were purchased from ATCC (Rockville, Md., USA). Authenticity of cell lines was confirmed at the DSMZ by STR (short tandem repeat) analysis, a PCR based DNA-fingerprinting methodology. Cell lines were routinely passaged once or twice weekly and maintained in culture for up to 20 passages. All cells were grown at 37° C. in a humidified atmosphere with 5% CO₂ in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.1 mg/mL gentamicin (Life Technologies, Karlsruhe, Germany).

The CellTiter-Blue® Cell Viability Assay (#G8081, Promega) was used according to manufacturer's instructions. Briefly, cells were harvested from exponential phase cultures, counted and plated in 96-well flat-bottom microtiter plates at a cell density of 6,000-20,000 cells/well depending on the cell line's growth rate. After a 24 hours recovery period to allow the cells to resume exponential growth, test compounds were added. Levormeloxifene or its pharmaceutically acceptable salts thereof was applied at 10 concentrations in 1.6-fold increments in duplicate and treatment continued for 96 hours. After 96 hours treatment of cells, 20 μL/well CellTiter-Blue® reagent was added. Following an incubation period of up to four hours, fluorescence (FU) was measured by using the Enspire Multimode Plate Reader (excitation λ=531 nm, emission λ=615 nm). For calculations, the mean values of duplicate/quadruplicate (untreated control) data were used. Sigmoidal concentration-response curves were fitted to the data points (T/C values) obtained for each cell line using 4 parameter non-linear curve fit (Oncotest Warehouse Software).

Levormeloxifene displayed concentration-dependent activity with sigmoidal concentration-effect curves in all cell lines tested. The geometric mean absolute IC₅₀ value for Levormeloxifene or its pharmaceutically acceptable salts thereof was determined as 1841 μM. Individual IC₅₀ values were in the range from 726 μM (HUP-T3) and 5352 μM (HPAC), corresponding to 7.4-fold difference between the most sensitive and most resistant cell line. Above-average sensitive cell lines (individual IC₅₀ values smaller than mean IC₅₀ value) were shown to be HUP-T3 (IC₅₀=726 μM), PA-TU-8902 (IC₅₀=1032 μM), Mia-Pa-Ca-2 (IC₅₀=1524 μM) and Panc-1 (IC₅₀=1545 μM).

TABLE 1
IC₅₀ values of Levormeloxifene and tamoxifen
in various pancreatic cancer cell lines

| | |
|---|---|
| levormeloxifene | TAM |

| | |
|---|---|
| Cell line | IC₅₀ (μM) |

| | | |
|---|---|---|
| Miapaca1 | 22 | 23 |
| Panc10.05 | 24 | 24 |
| Panc1 | 24 | 19 |
| BxPC3 | 21 | 19 |
| AsPC1 | 18 | 15 |
| SW1990 | 27 | 23 |

Thus it can be concluded that levormeloxifene shows considerable in vitro activity against pancreatic cancer.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

What is claimed is:

1. A method of treating pancreatic cancer comprising administering to a patient in need thereof an effective amount of levormeloxifene or a pharmaceutically acceptable salt thereof.

2. The method according to claim 1, wherein the pancreatic cancer is Stage 0, Stage IA, Stage IB, Stage IIA, Stage IIB, Stage III, or Stage IV.

3. The method according to claim 1, further comprising administering at least one other cancer therapy.

4. The method according to claim 3, wherein the additional cancer therapy comprises surgery, chemotherapy, immunotherapy, ablation, embolization, or ionizing radiation.

5. The method according to claim 4, wherein the additional cancer therapy comprises administering at least one additional chemotherapeutic agent comprising a nucleoside analog, antifolate, antimetabolite, topoisomerase I inhibitor, anthracycline, podophyllotoxin, taxanes, vinca alkaloid, alkylating agent, platinum compound, proteasome inhibitor, nitrogen mustard, estrogen analogue, monoclonal antibody, tyrosine kinase inhibitor, mTOR inhibitor, retinoid, immunomodulatory agent, histone deacetylase inhibitor, or other kinase inhibitor.

6. The method according to claim 5, wherein the additional chemotherapeutic agent comprises an antiestrogen.

7. The method according to claim 6, wherein the additional chemotherapeutic agent comprises tamoxifen, or a pharmaceutically acceptable salt thereof.

8. The method according to claim 4, wherein the additional cancer therapy comprises treatment with ionizing radiation.

9. The method according to claim 4, wherein the additional cancer therapy comprises surgery.

10. The method according to claim 1, wherein the patient has not been previously treated for pancreatic cancer.

11. The method according to claim 1, wherein the patient has undergone at least one prior treatment for pancreatic cancer.

12. The method according to claim 1, wherein the levormeloxifene is administered orally.

13. The method according to claim 1, wherein the levormeloxifene is administered in an amount from 50-500 mg/day.

14. A pharmaceutical composition comprising levormeloxifene and at least one other anti-cancer drug.

15. The composition of claim 14, wherein the anti-cancer drug is an antiestrogen.

16. The composition of claim 15, wherein the anti-cancer drug is tamoxifen, or a pharmaceutically acceptable salt thereof.

17. The method of claim 1, wherein the levormeloxifene is provided in a kit comprising levormeloxifene and instructions for a dosing regimen effective to treat pancreatic cancer.

18. A kit comprising levormeloxifene and instructions for a dosing regimen effective to treat pancreatic cancer, and at least one other anti-cancer drug.

19. The kit of claim 18, wherein the anti-cancer drug is tamoxifen or a pharmaceutically acceptable salt thereof.
